(19) United States
(12) Patent Application Publication
(10) Pub. No.: US 2026/0104387 A1
(43) Pub. Date: Apr. 16, 2026

(54) DEVICE AND METHOD FOR NATIVE SINGLE-CELL WESTERN BLOT

(71) Applicant: William Marsh Rice University, Houston, TX (US)

(72) Inventors: Julea VLASSAKIS, Houston, TX (US); Edgar Ernesto Ruiz BELLO, Houston, TX (US)

(73) Assignee: William Marsh Rice University, Houston, TX (US)

(21) Appl. No.: 19/359,425

(22) Filed: Oct. 15, 2025

Related U.S. Application Data
(60) Provisional application No. 63/707,416, filed on Oct. 15, 2024.

(51) Int. Cl. G01N 27/447; G01N 21/64; G01N 27/30; G01N 33/50; G01N 33/543; G01N 33/68

(52) U.S. Cl. CPC G01N 27/44756; G01N 21/64; G01N 27/30; G01N 33/5005; G01N 33/54386; G01N 33/6803; G01N 2474/10

(57) ABSTRACT

Apparatus and associated methods relate to a device for performing single-cell native western blotting. In an illustrative example, the device, for example, includes a microfluidic flow chamber. The microfluidic flow chamber may, for example, include a chamber configured to receive a micropatterned gel array. The microfluidic flow chamber may, for example, include opposing electrodes located on opposing sides of the chamber configured for electrophoresis. The microfluidic flow chamber may, for example, include a flow-in-port configured to receive buffer fluid into the chamber. The microfluidic flow chamber may, for example, include a flow-out-port configured to receive buffer fluid out of the chamber. Associated methods include a method of performing single-cell native western blotting. The method includes providing a patterned polyacrylamide gel slide formed by casting a polyacrylamide gel layer between a surface-treated microscope slide and a photoresist-patterned silicon wafer.

### CROSS REFERENCE TO RELATED APPLICATIONS

This application claims the benefit of U.S. Application Ser. No. 63/706,416, titled “Device and Method for Native Single-Cell Western Blot,” filed on Oct. 15, 2024.

This application incorporates the entire contents of the foregoing applications herein by reference.

### BACKGROUND

### 1. Field

Various embodiments generally relate to devices and methods for performing single-cell western blotting without denaturing conditions.

### 2. Description of Related Art

Current single-cell western blotting methods are not amendable to adaptation for measuring native proteins or protein complexes. Native single-cell Western blotting (scWB) is a technique that is used to analyze protein expression in individual cells while preserving their native, functional conformation. Unlike standard single-cell Western blotting, which denatures proteins using agents, native single-cell Western blotting employs non-denaturing conditions to maintain protein complexes and interactions.

### SUMMARY

Apparatus and associated methods relate to a device for performing single-cell native western blotting. In an illustrative example, an exemplary method includes providing a microfluidic flow chamber. The microfluidic flow chamber includes opposing electrodes located on opposing sides of the chamber configured for electrophoresis. The microfluidic flow chamber includes a flow-in-port and flow-out port configured to circulate buffer fluid into and out of the chamber. The method includes providing a micropatterned gel slide formed by casting a thin polyacrylamide gel between a surface-treated microscope slide and a photoresist-patterned silicon wafer. The method includes settling single cells into microwells of the micropatterned gel slide. The method includes lysing the cells in situ with non-denaturing cold buffer for a predetermined period configured such that native protein complexes are preserved without dissociation. The method includes applying electrophoretic field across the gel array for a predetermined period using the opposing platinum electrodes, configured such that protein complexes and multicomponent assemblies are not disassembled or separated by their size and charge. The method includes detecting one or more protein complexes and multicomponent protein assemblies.

In an illustrative example, the device, for example, includes a microfluidic flow chamber. The microfluidic flow chamber may, for example, include a chamber configured to receive a micropatterned gel array. The microfluidic flow chamber may, for example, include opposing electrodes located on opposing sides of the chamber configured for electrophoresis. The microfluidic flow chamber may, for example, include a flow-in-port configured to receive buffer fluid into the chamber. The microfluidic flow chamber may, for example, include a flow-out-port configured to receive buffer fluid out of the chamber. Associated methods include a method of performing single-cell native western blotting. The method includes providing a patterned polyacrylamide gel slide formed by casting a predetermined cell suspension on a slide configured such that the slide supports microwells for high-throughput single-cell analysis.

Some associated methods include stripping and sequentially probing the gel slide with additional antibodies for multiplexing, configured such that the multiple protein targets are sequentially quantified using fitted fluorescence data.

Some embodiments may, for example, fit the fluorescence peaks during the analysis. Some embodiments may, for example, use semi-automated image segmentation. Some embodiments may, for example, use machine learning-based feature analysis.

Some associated methods further include analyzing the fluorescence intensity profiles configured such that the data is fitted to quantify complex heterogeneity in single cells for therapeutic targeting.

Some associated methods include configurations wherein each microwell is seeded with at most one cell. Some associated methods may, for example, include performing the assay with purified protein solutions, instead of cells.

Some associated methods include configurations wherein the microwells have a diameter of 10-100 μm. Some associated methods include configurations, wherein the patterned polyacrylamide gel is about 40 μm thick.

Some associated methods further include flowing cold buffer through the chamber while applying the electrophoretic field to the gel. Some associated methods include recirculating cold buffer through the chamber while applying the electrophoretic field to the gel. Some associated methods include configurations, wherein the method preserves protein complexes and multicomponent protein assemblies.

Some embodiments include device configurations wherein the opposing electrodes include a platinum filament anode and a platinum filament cathode.

Some embodiments include device configurations wherein a free radical polymerization is used to make the polyacrylamide layer that is attached to the microscope slide. UV light may, for example, be used to crosslink the proteins to the gel.

Some embodiments include device configurations wherein the micropatterned gel array includes a series of columns and rows segments, wherein each segment is configured to receive a cell sample. Some embodiments may, for example, include segmentation with blocks or by region

Some embodiments include device configurations which further include opposing baffles configured to direct and regulate fluid flow of buffer fluid through the flow-in-port and flow-out-port.

Some embodiments include device configurations wherein the device includes an inlet buffer reservoir fluidly coupled to the flow-in-port; an inlet pump configured to pump buffer into the flow-in-port; an outlet pump configured to pump buffer out of the flow-out-port; and, an outlet buffer reservoir fluidly coupled to the flow-out-port configured to receive buffer out of the flow-out-port.

Some embodiments include device configurations which further include a buffer reservoir fluidly coupled to the flow-in-port and to the flow-out-port; and a pump fluidly coupled to the buffer reservoir configured circulate buffer fluid through the microfluidic chamber.

The pump may, for example, be a peristaltic pump. Some embodiments may, for example, include a temperature controller configured to regulate the buffer temperature. Some embodiments may, for example, include a flow controller configured to regulate the circulation of the buffer. Some embodiments may, for example, include a heat exchanger module configured to regulate the temperature of the buffer contained within the buffer reservoir. Some embodiments may, for example, include a power source coupled to the opposing electrodes to perform electrophoresis.

Before further describing various embodiments of the present disclosure in more detail by way of exemplary description, examples, and results, it is to be understood that the embodiments of the present disclosure are not limited in structure and application to the details as set forth in the following description. The embodiments of the present disclosure are capable of being practiced or carried out in various ways not explicitly described herein.

As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so.

Moreover, in the following detailed description, numerous specific details are set forth in order to provide a more thorough understanding of the disclosure. However, it will be apparent to a person having ordinary skill in the art that the embodiments of the present disclosure may be practiced without these specific details.

In other instances, features which are well known to persons of ordinary skill in the art have not been described in detail to avoid unnecessary complication of the description. While the present disclosure has been described in terms of particular embodiments, it will be apparent to those of skill in the art that variations may be applied to the apparatus and/or methods and in the steps or in the sequence of steps of the methods described herein without departing from the spirit and scope of the inventive concepts as described herein.

Any methods, devices and materials similar or equivalent to those described herein can be used in the practice or testing of the embodiments of the present disclosure. All such similar substitutes and modifications apparent to those having ordinary skill in the art are deemed to be within the spirit and scope of the inventive concepts as disclosed herein. The inclusion of any particular embodiment, feature or function within the Abstract is not intended to limit the scope of the present disclosure to such embodiment, feature or function. Titles and headings of sections of this disclosure are for convenience only and shall not affect the scope or interpretation of any aspect of this disclosure.

All patents, published patent applications, and non-patent publications referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the apparatus, methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.”

Where the specification or claims refer to “an additional” element, that does not preclude there being more than one of the additional element. That is, where the claims or specification refer to “a” or “an” element, such reference is not be construed that there is only one of that element. It is to be understood that where the specification states that a component, feature, structure, or characteristic “may”, “might”, “can” or “could” be included, that particular component, feature, structure, or characteristic is not required to be included.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Any embodiment of any of the present apparatus, methods, composition, kit, and systems may consist of or consist essentially of—rather than comprise/include/contain/have—the described steps and/or features.

Throughout this application, the terms “about” and “approximately” are used to indicate that a value includes the inherent variation of error for the apparatus or composition, the method used to administer the apparatus or composition, or the variation that exists among the components, objects, or study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately”, where used herein when referring to a measurable value of a dimension, quantity, or characteristic such as an amount, a temporal duration, thickness, width, length, density, and the like, is meant to encompass, for example, variations of ±20% or ±10%, or ±5%, or ±1%, or ±0.1% from the specified value, as such variations are appropriate to construct the apparatus or perform the disclosed methods and as understood by persons having ordinary skill in the art. As used herein, the term “substantially” means that the subsequently described event or circumstance completely occurs or that the subsequently described feature, event, or circumstance occurs to a great extent or degree. For example, the term “substantially” means that the subsequently described event or circumstance occurs at least 75% of the time, at least 80% of the time, at least 90% of the time, at least 95% of the time, or at least 98% of the time.

As used herein any reference to “one embodiment,” “an embodiment,” “some embodiments,” “particular embodiment,” or “a specific embodiment” or similar terminology means that a particular element, feature, structure, or characteristic described in connection with the embodiment is included in at least one embodiment. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the inventive concepts disclosed herein. Whether specifically noted as non-limiting examples or not, language describing examples, including “such as”, “including”, “other instances”, “merely exemplary”, “for instance”, “for example”, “etc.”, e.g.”, “as well as”, “the like”, and similar terms are understood to be non-limiting.

Variations of components and/or parameters discussed in relation to one embodiment described herein can be incorporated into other embodiments described herein. In non-limiting examples, ranges and related gradations for different temperatures, pressures, time, number of cycles, ratios, volumes, dimensions, current, voltage, fluorescence, brightness, and/or distances discussed in relation to one embodiment can also be incorporated into other embodiments disclosed herein. Additionally, in non-limiting embodiments, different configurations of components, including non-limiting examples such as pistons, chambers, cylinders, probes, sensors, power sources, detectors, lysis, and/or reagents discussed in relation to one embodiment can also be incorporated into other embodiments disclosed herein.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise. Other examples of changes, substitutions, and alterations are ascertainable by one skilled in the art and could be made without departing from the spirit and scope disclosed herein.

As used herein, “opposite” is used to indicate in a direction on average that is different to, orthogonal to, or opposing the general net vector of the first direction.

As used herein, when referring to movement of, or action on, a fluid, it is understood that this includes movement of, or action on, a fraction or subset of the fluid.

The inventive concepts of the present disclosure will be more readily understood by reference to the following examples and embodiments, which are included merely for purposes of illustration of certain aspects and embodiments thereof, and are not intended to be limitations of the disclosure in any way whatsoever. Those skilled in the art will promptly recognize appropriate variations of the apparatus, compositions, components, procedures and method shown below.

The details of various embodiments are set forth in the accompanying drawings and description below. Other features and advantages will be apparent from the description, and from the claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

Like reference symbols in the various drawings indicate like elements.

FIG. 1 depicts an exemplary CAD Design for an exemplary chamber.

FIG. 2 Schematic representation of an electrophoretic separation of native proteins and protein complexes from single cells in polyacrylamide gels.

FIG. 3 depicts an exemplary gel-slide system including a Polyacrylamide gel array casted on half a standard microscope slide.

FIG. 4A depicts an assembled flow chamber with buffer recirculation.

FIG. 4B depicts an assembled flow chamber without recirculation, discarding used buffer.

FIG. 5. depicts a demonstration how a dual-purpose lysis/run buffer can quickly increase the temperature and change the local pH near the electrodes inside the chamber with long separation times.

FIG. 6 depicts a native scWB in connection with a ladder and chaperone protein (Ewing Sarcoma).

FIG. 7 depicts native scWB assay preserving Ewing Sarcoma complexes for analysis to identify more than 1 complex in Ewing Sarcoma cells.

FIG. 8 depicts an exemplary comparison of single-cell SDS PAGE vs. Native PAGE.

FIG. 9 depicts an exemplary schematic of diffusion timescales of large complexes facilitating separation.

FIG. 10 depicts an exemplary assay validation using purified fluorescent complexes.

FIG. 11 depicts an exemplary flow chart for native single-cell Western blotting (scWB) for Ewing sarcoma protein analysis.

FIGS. 12-13 depicts exemplary analysis of the Joule heating impact on electrophoretic separations to require buffer exchange and recirculation.

FIG. 14 depicts high-throughput native EP with purified proteins to achieve high separation resolution.

FIG. 15 depicts an exemplary purified protein and analysis and HSP90α (1-3) cells, portions previously depicted in FIG. 7.

FIG. 16 depicts an exemplary dynamic interaction.

FIG. 17 depicts an exemplary expression and complex formation of heat shock proteins (HSPs) in heterogenous cell populations.

FIG. 18 depicts an exemplary microfluidic chamber system.

FIG. 19 depicts an exemplary diagram flow diagram prior to assembly.

FIG. 20 depicts an exemplary native scWB microfluidic chamber and operational system.

FIG. 21 depicts a comparison of low v. no flow conditions with purified and fluorescently labeled protein complexes.

FIG. 22 depicts a fluorescence micrograph of probed HSP90α on cancer cells TC-71.

FIGS. 23A-B depicts a representation band of TC-71 cells after immunoprobing for HSP90α, showing an intensity profile of both bands observed, and a correlation plot of AUC values for all measured cells.

### DETAILED DESCRIPTION

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### II. RELATED ARTS

Protein complexes and protein-protein interactions support cell homeostasis. Multimeric forms of protein complexes are often involved in signaling pathways, protein folding, gene regulation, and protein function. In disease, dysregulation of protein complexes can occur due to overexpression of interacting partners, protein recruitment in abnormal pathways, and more. In diseases with high cell-to-cell heterogeneity, such as cancer, understanding complexes expression and stoichiometric variation becomes crucial for designing novel therapeutic strategies and assessing current ones.

Currently, no technologies can measure multimeric protein complexes at the single-cell level with enough sensitivity and high-throughput to detect subpopulations. Accordingly, as described in this application a microfluidic approach may, for example, be used to spatially separate and measure protein complexes from individual cancer cells in 100s-1000s of cells.

For context, measurement of protein expression from individual cells can be achieved via mass spectrometry or immunoassays. Notably, single-cell western blots can multiplex detection of targets in 100s-1000s of cells simultaneously via a polyacrylamide based electrophoretic separation and in-gel immunoprobing. [2] In single-cell westerns, individual cells are deposited in a patterned polyacrylamide gel with well diameters tuned for the specific cell lines used. A quick lysis process, with denaturing and reducing conditions, limits diffusive protein losses within microwells. An electric field is quickly applied for 30-45s to size separate the proteins of interest, followed by a benzophenone-based photo immobilization of proteins to the gel network.

Despite the high sensitivity of mass spectrometry and the progress made in single-cell sample preparation, the measurements of protein complexes using this technique remains a challenge. In single-cell westerns, only small or denatured proteins can be measured. This is because denatured, SDS-coated proteins, or small proteins in native state can migrate fast through a gel network, maintaining electrolysis products and temperature variations to a minimum. For longer separation times, such as those required for large proteins or protein assemblies, Joule heating has been shown to increase diffusivity of the species, decreasing the available analyte and reducing separation resolution. [7]

Cytoskeletal complexes have been measured using a similar approach as single-cell westerns, where complex stabilizing and denaturing buffers are exchanged during single-cell electrophoretic assays using buffer-containing hydrogel lids. [5] Here, complexes and monomers are fractionated and analyzed independently. The number of protein species detectable at a time depends on the antibody species and fluorophores being used. For example, if all of your cytoskeletal targets of interest are detected with primary antibodies raised in a mouse (e.g., a testing animal), an observer will detect one protein at a time, and use the chemical stripping and re-probing to probe with the other mouse antibodies subsequently. Also, the method can fractionate multiple complexes composed of distinct proteins.

### III. Examples

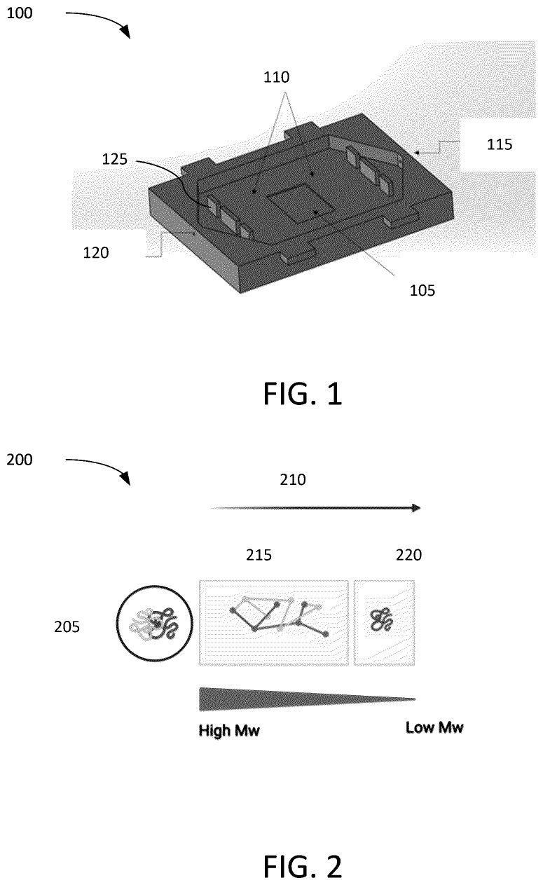

FIG. 1 depicts an exemplary CAD Design for an exemplary microfluidic chamber 100. The chamber may, for example, include a 3D printed chamber made of PLA. The chamber may, for example, be used to create the electrophoresis flow chamber by adding two platinum electrodes and electrode terminals made from steel shim. PLA filament may, for example, offer a cost-effective and customizable approach to fabricate intricate structures that facilitate precise electrophoresis flow. For example, the chamber may, for example, be printed with other filaments or with a resin-based 3D printer. Injection molding or other approaches for fabricating the chamber may, for example, be used.

Some slides that are received may, for example, include a polyacrylamide gel between a silicon wafer with an SU-8 pattern and a silinized microscope slide. After the gel is polymerized, cell suspension may, for example, be added on top of the gel to gravity settle the cells into the wells.

Electrophoresis in connection with flowing buffer may, for example, enable the separation of proteins from individual cells under controlled electric fields. Integrating platinum electrodes may, for example, offer stable and corrosion-resistant electrical conductivity. Platinum electrodes may, for example, ensure uniform voltage application without introducing contaminants during the lysis and separation phases. This configuration may, for example, optimize the electrophoresis flow dynamics and enhance resolution in protein detection and support high-throughput analysis of cellular heterogeneity.

Microfluidic chamber 100 houses the gel array receiver 105. Gel array receiver 105 may for example receive a slide including a gel array with micro wells that capture and isolate single cells for lysis and protein. Gel array receiver includes a recessed gap where the gel array would fit. For example, a patterned polyacrylamide gel holding single cells in individual microwell may, for example, be inserted into the gel array receiver. On opposing sides of gel array receiver 105, electrodes 110 are positioned to generate a uniform electric field. The uniform electric field may, for example, drive precise protein migration through the cell suspension during electrophoresis. Microfluidic chamber 100 includes a flow-in-port 115. The flow-in-port may, for example, channel the cell suspension, buffer, or reagents into the chamber directing them toward the gel array for processing. Microfluidic chamber 100 includes a flow-out-port. The flow-out-port 120 may, for example, remove waste or excess reagents ensuring efficient system clearance for high throughput. Microfluidic chamber 100 includes baffles 125. Baffles 125 may, for example, be configured to direct and regulate fluid flow of buffer fluid through the flow-in-port and flow-out-port.

Native single-cell western blotting builds on denaturing single-cell westerns and immunoassays, however, a novel electrophoresis chamber design and electrophoresis conditions make possible the preservation and efficient separation of large molecular weight species without compromising separation resolution and minimizing diffusive losses.

The native electrophoretic separation of protein complexes requires mild lysis conditions that allow preservation of multimeric structures while completely lysing the cell membrane. As mentioned above, previous studies reported lysis buffer compositions for denaturing and reducing conditions, as well as cytoskeletal complexes. A dual-purpose buffer formulation [5] that solubilizes the cytosolic proteins and is used as a conductive buffer that permits electrophoresis.

Constant cold lysis and electrophoresis conditions are necessary to preserve protein assemblies. Elevated buffer temperatures that arise from Joule heating caused by high electric fields and buffers with high conductivity can quickly lead to increased diffusive losses and decreased separation resolution. [9] Low conductivity buffer replacements have been shown to decrease Joule heating [7], however electrophoresis products still accumulate near the electrodes leading to local pH variations that influence the mobility of the protein species. Here, a constant flow of a chilled (4° C.) dual purpose lysis/run buffer was used during the longer separation times (3-5 mins), when compared to denaturing conditions (30-45s) for individual protein species.

To achieve electrophoresis under a buffer flow, a flow and electrophoresis microfluidic chamber was used to control the electric field while maintaining a constant temperature and preventing the accumulation of electrolysis products. The chamber may, for example, be 3-D printed. A polylactic acid (PLA) chamber may, for example, be used and equipped with platinum electrodes that can interface with a commercial electrophoresis power supply. The chamber may, for example, be coupled to a peristaltic pump that circulates chilled buffer from a reservoir.

FIG. 2 depicts a schematic representation 200 of an electrophoretic separation of native proteins and protein complexes from single cells in polyacrylamide gels. Schematic representation 200 includes a cell 205. Cell 205 is isolated in the cell suspension. The cell suspension may, for example, be gravity settled to ensure precise positioning before lysis to release protein content. Protein within cell 205 migrates along a migration direction 210 from high Mw (molecular weight) to low Mw. Migration direction 210 affects multicomponent high Mw region protein complexes 215 to migrate to lower Mw. Native protein complexes 220 are affected by the migration direction 210.

For example, in order to separate native proteins and protein complexes, single cells were gravity settled in a patterned polyacrylamide gel (˜40 μm thick). The microwells in the gel have diameters that can be adjusted based on the size of the cells of interest (˜10-100 μm). The pattern is first imprinted via photolithography onto a silicon wafer, then used as a mold for the polyacrylamide gel.

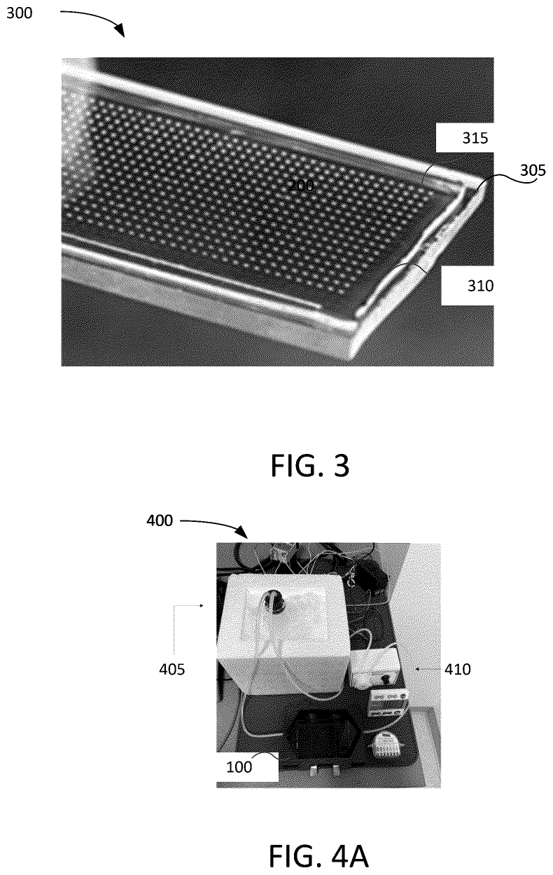

FIG. 3 depicts an exemplary gel-slide system 300 including a polyacrylamide gel array cast on half a standard microscope slide. Gel-slide system 300 includes a slide 305. Slide 305 may, for example, serve as the foundational substrate. Gel 310 is cast on slide 305 to capture cells and facilitate separation. Single cells 315 are gravity settled and uniformly spaced (e.g., columns and rows) into an array extending across the width and length of the gel. The flow chamber was designed to fit a gel cast on half a standard microscope slide (37.5×25 mm) (FIG. 3).

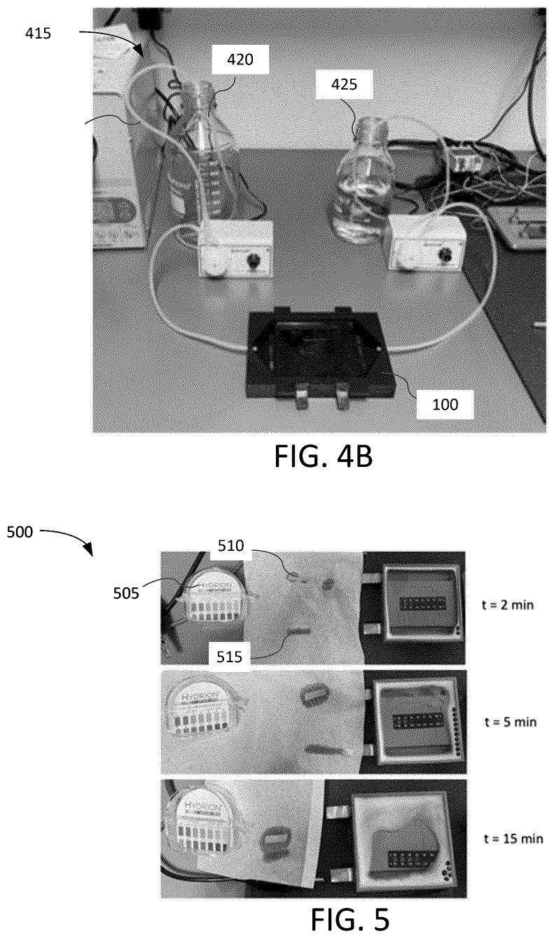

FIG. 4A depicts an assembled electrophoresis flow chamber 400 with buffer recirculation. A heat exchanger system may, for example, be used to maintain the desired temperature throughout the electrophoresis assay time. Assembled electrophoresis flow chamber 400 uses cold buffer from a reservoir and flows it through chamber 100. A heat exchanger 405 maintains cold buffer at low temperatures to minimize protein degradation during lysis and electrophoretic separation.

For context, cold buffer may, for example, include a chilled solution used in single-cell Western blotting may, for example, be used to maintain a stable pH and ionic environment. A peristaltic pump 410 delivers and circulates fluid into and out of chamber 100. Gravity settling is performed outside of the chamber without buffer flow. While an electric field separates proteins in the gel array, the cold buffer flow avoids local pH gradients that could occur near the electrodes over long times (>45 sec) if no flow is present, as well as maintaining a low temperature in the chamber to limit Joule heating effects and preventing temperature from inducing complex dissociation or protein denaturation. The initial tests of the flow chamber were made using either one or two peristaltic pumps, to recirculate buffer or flow it through the chamber and then to discard used buffer.

FIG. 4B depicts an exemplary system 415 with assembled flow chamber 100 without recirculation, discarding used buffer. Exemplary system 415 includes unused buffer 420. Exemplary system 415 includes used buffer 425. The unused buffer may, for example, be pumped into the microfluidic chamber. Excess buffer may, for example, be pumped into the used buffer reservoir.

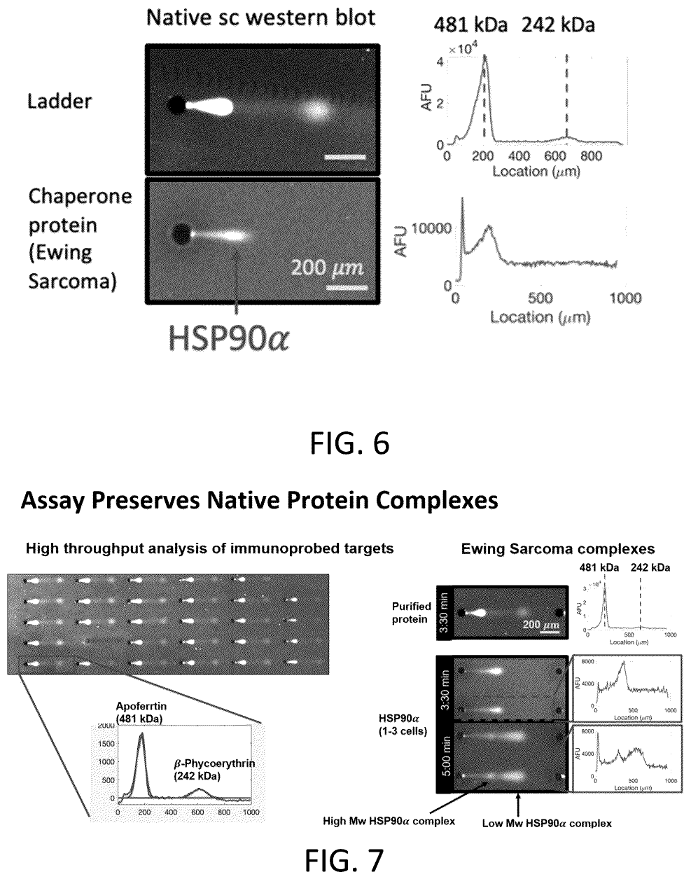

The system as depicted in FIGS. 4A and 4B may, for example, be used to perform electrophoresis to separate native complexes with controlled pH and temperature. The native complexes were distinguished in their native state by using a two-protein ladder, composed of two fluorescently labeled proteins of 242 and 481 kDa (β-phycoerytrhin and Apoferritin, respectively, FIG. 6). The protein solution was composed of two multimeric protein complexes. The technology was further tested using a cancer cell line (Ewing Sarcoma, TC-71) and protein injection and one band of the chaperone protein HSP90α was observed. HSP90α is known to form multi-component, high molecular weight protein complexes. The denatured monomeric form of HSP90α is 90 kDa. Heat shock proteins are examples of proteins that form complexes or interact with each other. It is shown later that it is possible to distinguish two bands that correspond to hsp90α complexes.

FIG. 5. depicts a demonstration of how a dual-purpose lysis/run buffer can quickly increase the temperature and change the local pH near the electrodes inside the chamber with long separation times. During testing it was discovered that a clear local pH change occurred near the electrodes, resulting from the creation of electrolysis products. FIG. 5 depicts a pH-sensitive fluorescent dyes 505. A pH-sensitive fluorescent dye strip 510 and pH-sensitive strip 515 depicts the pH changes over time.

FIG. 6. depicts preliminary results of the native single-cell western blot. (Left). FIG. 6 depicts a false-colored micrograph of fluorescently labeled ladder and immunoprobed HSP90α. (Right). The false-colored micrograph depicts the fluorescence intensity profiles of protein ladder and HSP90α.

FIG. 7 depicts native scWB assay preserving Ewing sarcoma complexes for analysis. Conditions were optimized and the results where able to identify more than 1 complex in Ewing Sarcoma cells. Electrophoretic separation showing bands of a purified protein solution ladder and endogenous HSP90alpha (a chaperone protein) from Ewing Sarcoma cells, with associated fluorescence intensity profiles. Additionally, the predetermined “immunoprobed targets” are depicted in the bottom right micrographs. The images to the left and top right are fluorescently labeled proteins.

Native scWB may, for example, enable high throughput analysis of immunoprobed targets of Ewing sarcoma complexes. The process may, for example, include separating high Mw HSP90α complexes from low Mw HSP90α complexes. For context immunoprobed targets are specific proteins, like for example HSP90α in Ewing Sacoma. Immunoprobed targets may, for example, be detected using fluorescently labeled antibodies in native sing-cell Western blotting to analyze their expression and interactions. The kilodalton (kDa) is a unit of molecular mass which may, for example, be used to quantify the size of protein of complexes in electrophoretic separation. The analysis may, for example, be used to reflect migration behavior in polyacrylamide gels.

Some embodiments may, for example, include a native single-cell Western blotting (scWB) system for analyzing Ewing sarcoma protein complexes. In some embodiments of scWB systems, an AFU (arbitrary fluorescence intensity) and location (μm) graph may, for example, be used to quantify fluorescence intensity compared to the position along the migration direction.

For context, Ewing sarcoma (ES) and single-cell Western blotting (scWB), protein expression dictates cell fate by orchestrating a cascade from DNA to protein, where dynamic interactions and altered molecular networks drive tumor heterogeneity and therapeutic outcomes. DNA may, for example, encode genes, such as the EWSRI-FLI fusion in ES, which undergoes transcription to produce RNA, followed by translation into proteins like EWS-FLI1 oncoprotein and its chaperone HSP90. [4].

For example, native single-cell Western Blotting (scWB) in microfluidic chamber 100 may, for example, reservoir Ewing sarcoma protein complexes for high-throughput single-cell analysis. Fast lysis with cold buffer in connection with the flow-in-port 115 may, for example, ensure rapid protein release without degradation. Temperature control using heat exchanger 405 and cold buffer may, for example, prevent complex dissociation during electrophoresis with platinum electrodes. pH control via pH-sensitive strips may, for example, be used to ensure stability to avoid denaturation. This may, for example, support precise ES heterogeneity analysis.

Heterogenous expression of multimeric complexes in Ewing sarcoma analyzed via native scWB in microfluidic chamber 100 may, for example, use 481 and 242 kDa purified complexes (481 kDA, 170 kDa) across single cells. AFU/location graphs from scWB may, for example, show diverse fluorescent profiles to indicate heterogenous oncogenic signaling. These networks may, for example, promote proliferation and immune evasion in Ewing sarcoma. The single cell analysis may, for example, identify therapeutic targets to disrupt these complexes.

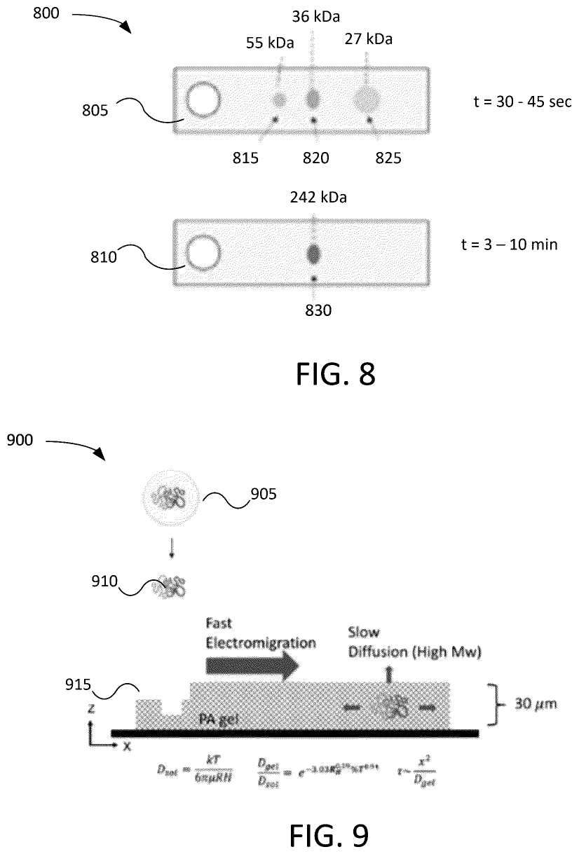

FIG. 8 depicts an exemplary comparison 800 of single-cell SDS PAGE vs. Native PAGE. Comparison 800 depicts a single cell SDS PAGE assay 805 compared to a Native PAGE assay 810. Single cell SDS PAGE assay 805 depicts the results after a rapid lysis 30-45s. Single cell SDS PAGE assay 805 includes β-Tub 815, 55 kDa. Single cell SDS PAGE assay 805 includes glyceraldehyde-3-phosphate dehydrogenase (GAPDH 820), 36 kDa. Single cell SDS PAGE assay 805 includes enhanced green fluorescent protein 825 (EGFP), 27 kDa. Native PAGE assay 810 includes a HSP90 dimer 830 (cancer-associated), 242 kDa, The 3-10-minute time frame reflects the time needed for effective separation of high molecular weight complexes (e.g., 242 kDA) in native PAGE, to enable analysis of heterogenous protein networks for therapeutic targeting.

ScWB may, for example, be used to separate denatured proteins in short times. ScWB presents limitations for native separation due to longer times and buffer chemistries needed. For example, joule heating negatively impacts long electrophoresis (EP) times. A positive correlation between EP times and temperature exists. A positive correlation between EP times and pH gradients exist. A positive correlation between complex dissociations exists. For example, temperature as a function of run time with different buffers in open microfluidic electrophoresis may, for example, increase over time (e.g., EP time).

FIG. 9 depicts an exemplary schematic 900 of diffusion timescales of large complexes facilitating separation. The following formulas are used to quantify protein diffusion in gel (D gel) vs. diffusion in solution (Dₛₒₗ) using constants for gel concentration, protein size, thermal energy, and viscosity.

$${D}_{sol} = \frac{k T}{6 π RH} \qquad ( 1 )$$

$$\frac{{D}_{gel}}{{D}_{sol}} = {e}^{- 3.03 {R}_{H}^{0.59}} {T}^{\text{.94}} \qquad ( 2 )$$

$$τ = \frac{{x}^{2}}{{D}_{gel}} \qquad ( 3 )$$

Where τ is the characteristic diffusion time (in seconds) for proteins (e.g., HSP90, β-tub, GAPDH, EGFP) to diffuse a specific distance x (e.g., in micrometers) through the polyacrylamide gel represented by the diffusion coefficient (D_{gel}).

Exemplary schematic 900 includes mild lysis conditions 905. In the native sing-cell Western blotting (scWB) assay 915 for Ewing sarcoma mild lysis conditions 905 may, for example, be used to preserve protein-protein interactions 910. For example, fast electromigration and slow diffusion in a 30 μm polyacrylamide (PA) gel preserves protein-protein interactions 910 in the X—Z plane.

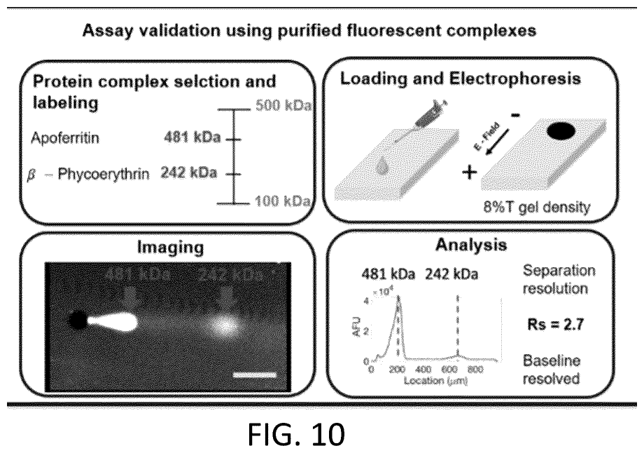

FIG. 10 depicts an exemplary assay validation using purified fluorescent complexes. For example, in validating the native single-cell Western blotting (scWB) assay for Ewing sarcoma within a 3D-printed microfluidic chamber, purified fluorescent protein complexes, ferritin (481 kDa) and β-phycoerythrin (240 kDa) were selected to assess separation performance. These complexes were loaded with a solution containing purified and fluorescently labeled protein complexes (+10% glycerol) were deposited on top of the gel array. This experiment illustrated the separation performance with known protein concentrations and without lysis and immunoprobing followed by electrophoresis. Fluorescent imaging captures distinct bands, with AFU/location graphs being used to show the baseline-resolved peaks (e.g., the Rₛ=2.7), indicating clear separation of ferritin and β-phycoerythrin based on their molecular weight. The analysis confirms high-throughput quantification of complex migration, validated against know standards, ensuring the assay's precession for resolving heterogenous protein complexes like HSP90α in Ewing sarcoma for therapeutic targeting.

For context, purified proteins were sourced and labelled with dyes. Endogenous proteins were from cancer cells were immunoprobed. Wherever there is HSP90 protein in the text or images, it is always endogenous.

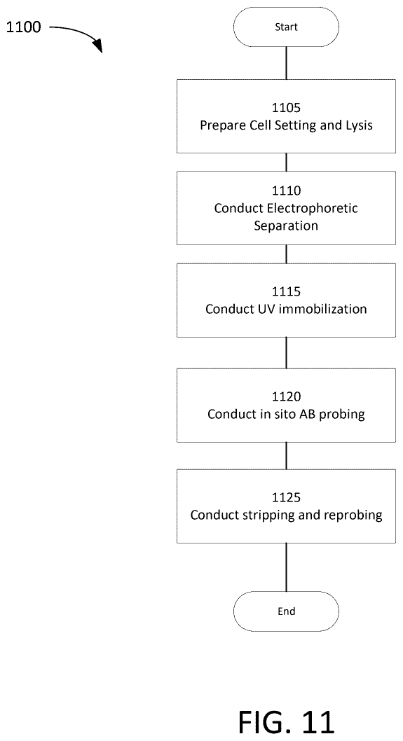

FIG. 11 depicts an exemplary flow chart 1100 for native single-cell Western blotting (scWB) for Ewing sarcoma protein analysis. In step 1105, a user of the method prepares cell setting and lysis with cold buffer for predetermined time (e.g., 30-45 s). In step 1110, the user of the method conducts electrophoretic separation (e.g., 3-10 min) using platinum electrodes. In step 1115, the user of the method performs UV immobilization to immobilize proteins with the cured resin. In step 1120, the user of the method probes in sito with fluorescent antibodies. In step 1425, the user of the method strips and reprobes for multiplexing.

For the electrophoresis and flow chamber assembly, two platinum electrodes may, for example, be placed inside a PLA 3D printed chamber, spaced by a 4 cm gap. Both electrodes may, for example, be soldered to pieces of steel shim, which can be connected to an electrophoresis power supply (available by BioRad, Hercules, California).

A peristaltic pump may, for example, be used to recirculate buffer inside the chamber by connecting a flexible tube to opposite sides of the chamber. The chamber may, for example, be designed to have a flow settling zone on one side, to reduce the initial jetting of the flow and ensure an even distribution of buffer through the surface of the gel array that is placed inside the chamber. A 1 L buffer reservoir is placed inside an insulated container with ice and water to maintain the buffer near 4° C. throughout the assay.

The polyacrylamide gel array fabrication may, for example, be conducted using SU-8 photolithography to create an array of cylindrical microposts on a silicon wafer. The dimensions of the posts are chosen based on an empirically optimized aspect ratio of 1.3. [1] The wafer is silanized and used to cast 8-10% T polyacrylamide gels on half standard size microscope slides.

Cell loading may, for example, be conducted as follows using a 100 μl of a single-cell suspension with a density of 1×10⁶ cells/ml deposited on each microarray half slide to analyze. Slides are shaken every 2 mins for 10 mins to maximize single cell loading. 3×1 ml PBS washes are used to remove excess cells and a 1×1 ml Tris-HCl PH=7.5 wash is used to equilibrate the gel pH.

The buffer composition may, for example, include dual purpose lysis/run buffer: 0.1× tris-HCl PH=7.5, 2 mM MgCl2, 1% v/v Triton X-100.

The electrophoresis may, for example, include configurations where the gel array is loaded into microfluidic chamber 100. Lysis is performed by using cold dual-purpose lysis/run buffer for 1:30 min, followed by a 30 sec injection step, performed under no flow conditions. Recirculation is then started for 3-5 mins, while the electric field is present. Electric fields of 30-40 V/cm ensure both a rapid separation and joule heating that can be controlled using the proposed buffer recirculation system.

Photoimmobilization may, for example, be conducted using benzophenone methacrylamide group incorporated into the polyacrylamide gel precursor solution allowing a 45 s UV irradiation of the gel array inside of the electrophoresis chamber, that ensures crosslinking of the separated proteins to the gel, preventing diffusive losses during the analysis steps.

Immunoprobing may, for example, be conducted using 50-100 μl of an antibody dilution is pipetted between a glass plate and the gel array facing down. This technique allows the antibody to diffuse to the gel via capillary action and minimize the volume required. Similar to conventional western blotting, the gel arrays are probed with a primary antibody selected for the target of interest (2 hr probing), as well as with a secondary fluorescently labeled antibody (1 hr probing). 3×10 min TBST washes are performed to remove excess antibody. [1]

Analysis may, for example, be conducted with the gel arrays. The gel arrays may, for example, be scanned using a commercial microarray scanner (Genepix 4300, available in Elmwood Park, New Jersey) and micrographs are analyzed using the SUMMIT pipeline. [6] Each microwell associated with an individual cell is associated with a region of interest (ROI), where the fluorescent intensity and signal-to-noise ratio are measured.

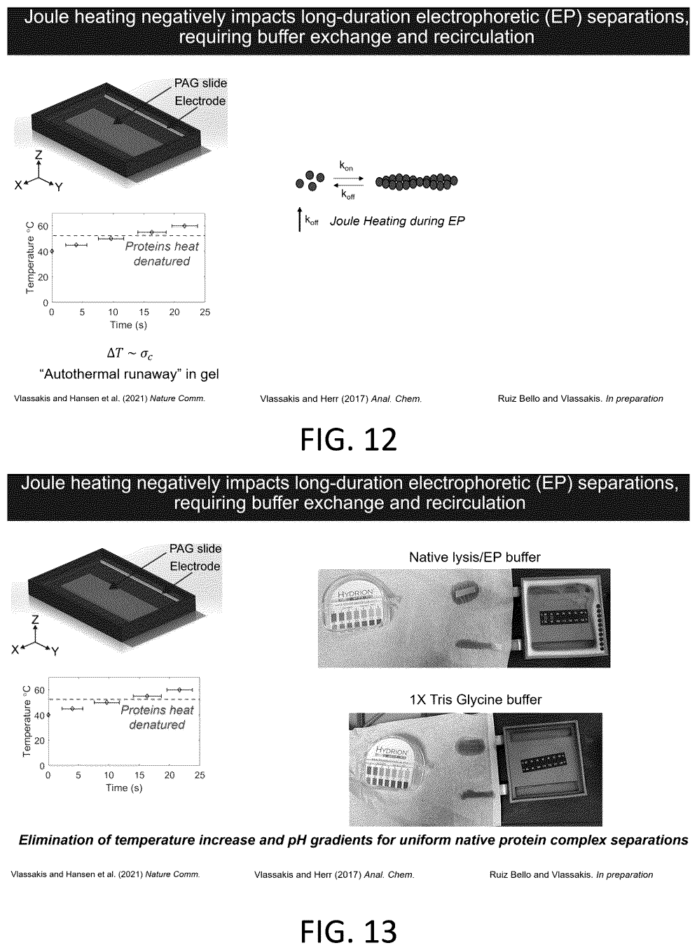

FIGS. 12-13 depict exemplary analysis of the Joule heating impact on electrophoretic separations to require buffer exchange and recirculation. Joule heating during long-duration electrophoretic (EP) separation in a polyacrylamide (PA) slide negatively impacts protein stability risking thermal runaway in the gel. Platinum electrodes exacerbate heat buildup, necessitating buffer exchange and recirculation to maintain temperature control and ensure reliable separation of complexes. Eliminating temperature and pH gradients may, for example, be important for uniform separation of native protein complexes.

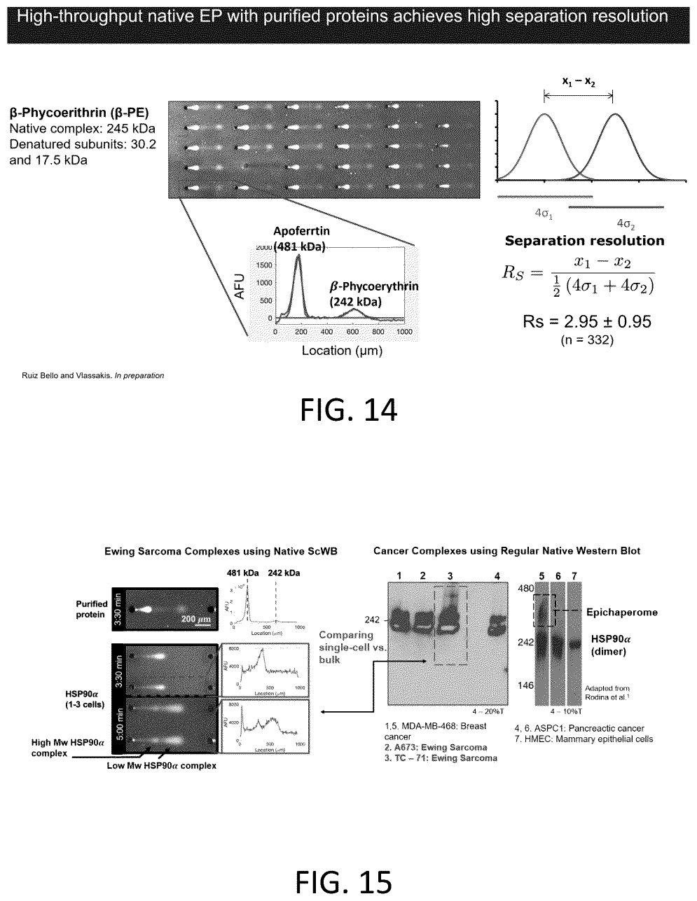

FIG. 14 depicts high-throughput native EP with purified proteins to achieve high separation resolution. Purified proteins like ferritin and β-phycoerythrin undergo native EP in the microfluidic chamber. Single cells settle in the polyacrylamide gel and electrophoresis is used. The resolution may, for example, be determined with the following equations.

$${R}_{S} = \frac{{x}_{1} - {x}_{2}}{\frac{1}{2} ( 4 {σ}_{1} + 4 {σ}_{2} )} \qquad ( 4 )$$

$${R}_{s} = 2.95 \pm 0.95 \qquad ( 5 )$$

Where n=332, representing the number of independent single-cell measurements or replicated used to calculate the mean separation resolution (Rₛ=2.95±0.95) for native electrophoresis of purified protein complexes in the scWB assay.

FIG. 15 depicts measuring chaperone protein complexes in cancer cells (bulk). The right image depicted being compared is from FIG. 1d that shows a western blot.[3] Measuring chaperone protein complexes in bulk cancer cell populations uses traditional native Western blotting to detect multi-component assemblies but averages the signals and masks heterogeneity. Native single-cell Western blotting (scWB) may, for example, outperform bulk analysis by resolving these complexes in single cells with high resolution. This may, for example, enable precise quantification of chaperone interactions critical for Ewing sarcoma therapeutic targeting.

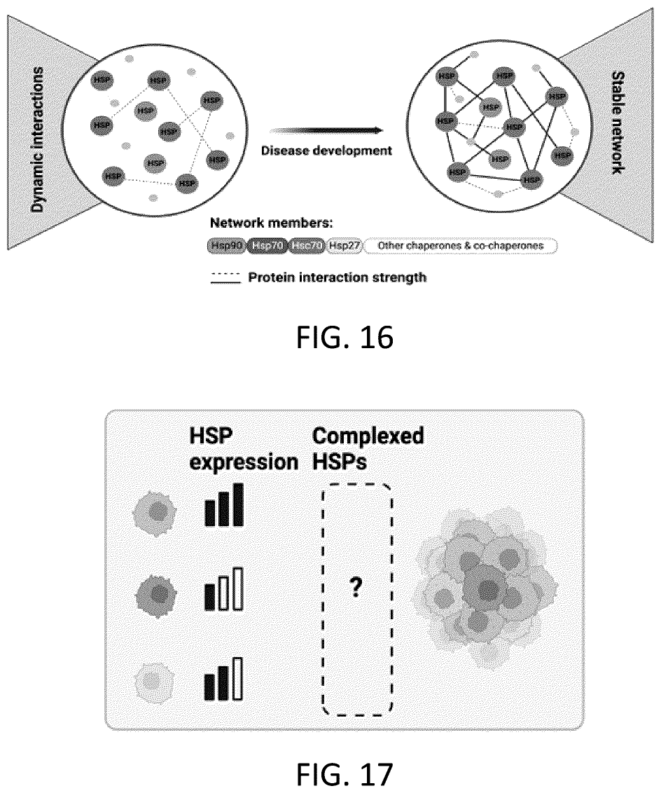

FIG. 16 depicts an exemplary dynamic interaction and FIG. 17 depicts an exemplary expression and complex heat shock proteins (HSPs.). Dynamic interactions of network members such as Hsp90, Hsp70, Hsc70, Hsp27, and other chaperones & co-chaperones) may, for example, Lead to changes in interactions strengths or stoichiometries that could be associated with disease. As depicted in FIG. 17, quantifying interaction strengths for complex HSPs remain challenging. Incomplete identification of network members, chaperones, co-chaperones, or interacting proteins may, for example, hinder quantitative assessment.

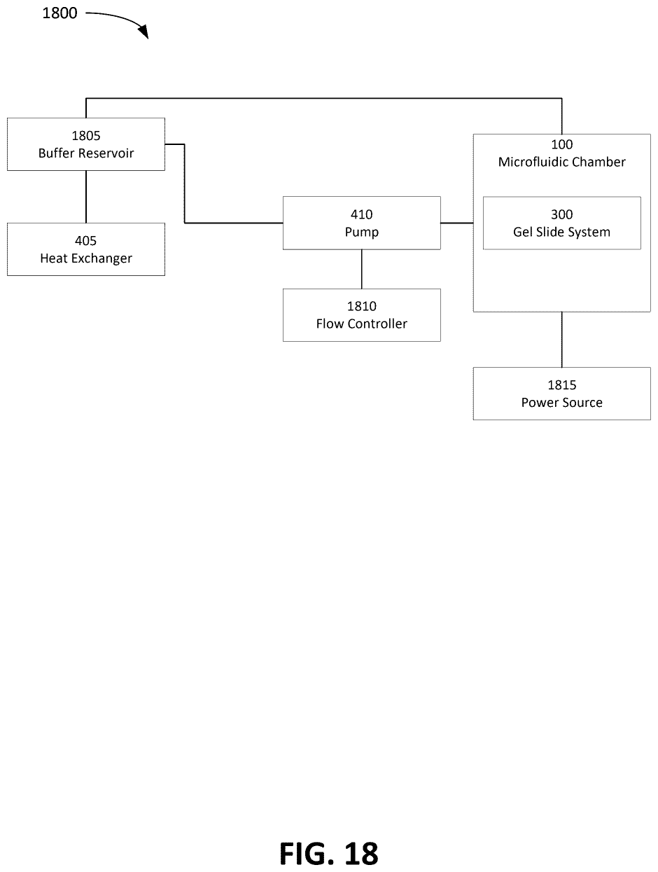

FIG. 18 depicts an exemplary microfluidic chamber system 1800. Microfluidic chamber system 1800 includes a buffer reservoir 1805. Buffer reservoir is fluidly coupled to heat exchanger 405. Buffer reservoir 1805 is fluidly connected to pump 410. Pump 410 is controlled by a flow controller 1810. The controller may, for example, be used to change the pump rate. Pump 410 is fluidly connected to the microfluidic chamber 100. Microfluidic chamber 100 includes a gel slide system. Microfluidic chamber 100 includes electrodes which are connected to a power source 1815 to conduct electrolysis on the gel slide system 300, while cold buffer is recirculated in to and out of the microfluidic chamber.

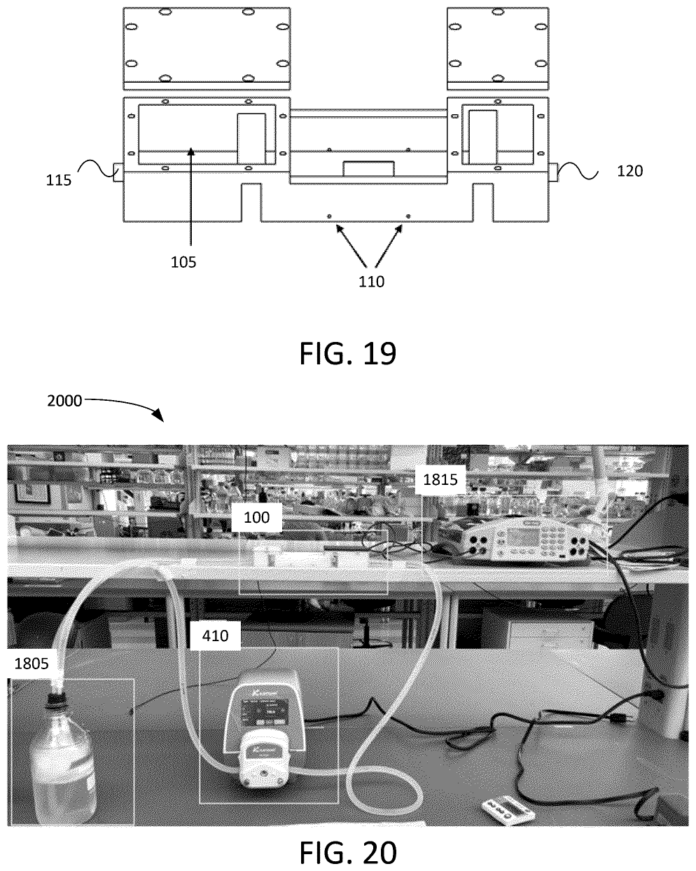

FIG. 19 depicts an exemplary diagram flow diagram prior to assembly. FIG. 25 depicts an exemplary native scWB microfluidic chamber system 2000. Native scWB chamber system 2000 includes electrophoresis microfluidic chamber 100. Native scwb chamber 2000 includes power source 1815 (e.g., BioRad power supply). Native scWB chamber includes buffer reservoir 1805. Native scwb chamber 2000 includes pump 410 (e.g., a. peristaltic pump). Pump 410 is coupled to buffer reservoir 2005.

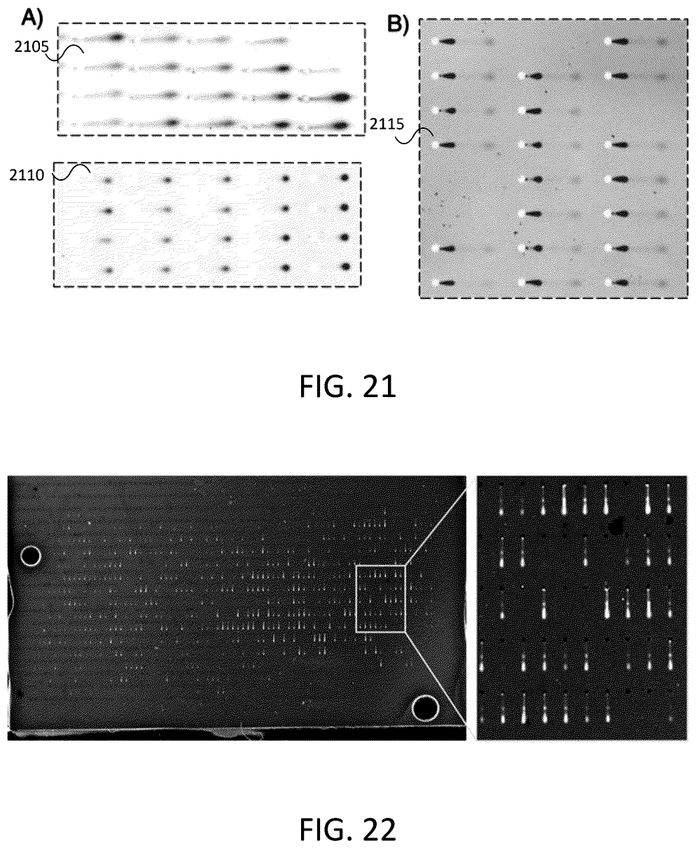

FIG. 21 depicts a comparison of flow 2110 v. no flow conditions 2105 with purified and fluorescently labeled protein complexes. For example, using a recirculation chamber, observations were made how diffusive losses were limited and band quality was improved by using purified and fluorescently labeled protein complexes. The purified protein solutions mixed with 10% glycerol and incubated on top of gel array for 5-7 mins. The excess solution was removed, and the array was placed inside of the electrophoresis chamber. An initial injection step with no flow limited losses, followed by a buffer recirculation and electrophoresis step. First-phycoerythrin (242 kDa), a multimeric complex, was used to test flow and no-flow conditions, observing clear differences in peak width. Bands that resulted from flow conditions had an average peak width of 73.8 μm (CV=24.4%) and average migration distance of 565.2 μm (CV=8.0%, n=1003). Under no flow conditions, the average peak width was 210.6 μm (CV=27.5%) and a migration distance of 604.0 μm (CV=13.7%, n=816). This resulted in a statistically significant reduction of the band widths (one tailed F-test=10.35, p<0.001).

FIG. 21 depicts a mixed flow 2115 of the two protein complexes Apoferritin (481 kDa) and β-phycoerythrin mixed together, to test the separation efficiency of native scWB. A separation resolution of resolution (Rs) of 2.95±0.95 (n=332) was observed using 3:30 min run time and 33V/cm. For context, these results were obtained using purified protein complexes, and these are also depicted in FIGS. 6, 7, and 14, where it mentions ladder or purified protein. The first band corresponds to Apoferritin and the second band to beta-phycoerythrin.

FIG. 22 depicts a fluorescence micrograph of probed HSP90α on cancer cells TC-71. Endogenous complexes were resolved from single cancer cells: Using similar conditions as the ones described for purified protein solutions, pediatric cancer cell line TC-71 was selected to probe for the molecular chaperone HSP90α, involved in the formation of high MW multimeric complexes, that have also been associated to aberrant complexes⁶. We found two complexes using a separation time of 3:00 min and an electric field of 40 V/cm.

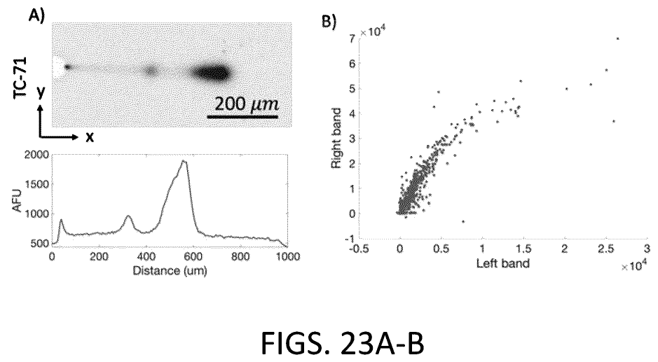

FIG. 23A depicts a representation band and correlation plot of AUC values for all measured cells. FIG. 23B includes a correlation plot of AUC values for all cell measured. The plotted intensity profile suggests higher detectable abundance of a lower MW complex and a second complex with lower abundance and higher MW. A correlation plot comparing the area under the curve (AUC) for each peak in all the cells analyzed shows a positive non-linear correlation, where doublets and triples could lie on the higher end of both axes. Some embodiments, may, for example, prove the assay can measure multicomponent assemblies. The expected signal may, for example, overlap. Different fluorophores may, for example, be used to identify the multiplexed signal.

Although various embodiments have been described with reference to the figures, other embodiments are possible.

Although exemplary apparatuses, systems, and methods have been described with references to the figures, other implementations may be deployed in other industrial, scientific, medical, commercial, or residential applications.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by references:
1. Kang, C., Single cell-resolution western blotting. Nat Protoc. 11(8) 1508-30 (2016). doi: 10.1038/nprot.2016.089.
2. Hughes, A., et al. Single-cell western blotting. Nat Methods 11, 749-755 (2014). doi.org/10.1038/nmeth.2992.
3. Rodina, A. et al. Nature 538, 397-401 (2016).
4. Wang, T. et al. J Biol Chem 294, 2162-2179 (2019).
5. Vlassakis, J. et al. Nat Commun 12, 4969 (2021).
6. Vlassakis, J. et al. SLAS Techol 26, 637-649 (2021).
7. Vlassakis, J. & Herr, A. Anal Chem 89, 12787-12796 (2017).
8. Updyke, Timothy, and Thomas Beardslee. Compositions and Methods for Improving Resolution of Biomolecules Separated on Polyacrylamide Gels. U.S. Pat. No. 9,057,694B2, 16 Jun. 2015.
9. Dutta, T. & Vlassakis, J. Microscale measurements of protein complexes from single cells. Curr. Opin. Struct. Biol. 87, 102860 (2024).

What is claimed:

1. A method of performing single-cell native western blotting, the method comprising
providing a microfluidic flow chamber comprising:
a chamber configured to receive a micropatterned gel slide comprising microwells;
opposing electrodes located on opposing sides of the chamber configured for electrophoresis;
a flow-in-port configured to receive buffer fluid into the chamber;
and,
a flow-out-port configured to receive buffer fluid out of the chamber;
providing a micropatterned gel slide formed by casting a polyacrylamide gel layer between a surface-treated microscope slide and a photoresist-patterned silicon wafer;
settling single cells into microwells of the micropatterned gel slide;
lysing the cells in situ with non-denaturing cold buffer for a predetermined period configured such that native protein complexes are preserved without dissociation;
applying electrophoretic field across the micropatterned gel slide for a predetermined period using the opposing platinum electrodes, configured such that protein complexes and multicomponent assemblies are not disassembled or separated by their size and charge; and,
detecting one or more protein complexes and multicomponent protein assemblies.

2. The method of claim 1, further comprising: stripping and sequentially probing the gel slide with additional antibodies for multiplexing configured such that the multiple protein targets are sequentially quantified using fitted florescence data.

3. The method of claim 1, further comprising analyzing the fluorescence intensity profiles configured such that the data is fitted to quantify complex heterogeneity in single cells for therapeutic targeting.

4. The method of claim 1, wherein each microwell is seeded with at most one cell.

5. The method of claim 1, wherein the microwells have a diameter between 10 μm to 100 μm.

6. The method of any one of claim 1, wherein the polyacrylamide gel layer is about 10-100 μm thick.

7. The method of any one of claim 1, further comprising flowing cold buffer through the chamber while applying the electrophoretic field to the gel.

8. The method of any one of claim 1, wherein the method preserves protein complexes and multicomponent protein assemblies.

9. A device for performing single-cell native western blotting, the device comprising:
a microfluidic flow chamber comprising:
a chamber configured to receive a micropatterned gel slide comprising microwells;
opposing electrodes located on opposing sides of the chamber configured for electrophoresis;
a flow-in-port configured to receive buffer fluid into the chamber; and,
a flow-out-port configured to receive buffer fluid out of the chamber.

10. The device of claim 9, wherein the opposing electrodes comprise a platinum filament anode and a platinum filament cathode.

11. The device of claim 9, wherein the opposing electrodes are soldered to pieces of steel shim connected to an electrophoresis power supply.

12. The device of claim 9, wherein the micropatterned gel slide is formed by casting a polyacrylamide gel layer between a surface-treated microscope slide and a photoresist-patterned silicon wafer.

13. The device of claim 9, wherein the predetermined cell suspension comprises a series of columns and rows segments, wherein each column and row segment is configured to receive a cell sample.

14. The device of claim 9, further comprising opposing baffles configured to direct and regulate fluid flow of buffer fluid through the flow-in-port and flow-out-port.

15. The device of claim 9, further comprising:
an inlet buffer reservoir fluidly coupled to the flow-in-port;
an inlet pump configured to pump buffer into the flow-in-port;
an outlet pump configured to pump buffer out of the flow-out-port; and,
an outlet buffer reservoir fluidly coupled to the flow-out-port configured to receive buffer out of the flow-out-port.

16. The device of claim 9, further comprising:
a buffer reservoir fluidly coupled to the flow-in-port and to the flow-out-port; and,
a pump fluidly connected to the buffer reservoir configured circulate buffer fluid through the microfluidic chamber.

17. The device of claim 16, wherein the pump is peristaltic pump.

18. The device of claim 16, further comprises a flow controller configured to regulate the circulation of the buffer.

19. The device of claim 16, further comprises a heat exchanger module configured to regulate the temperature of the buffer contained within the buffer reservoir.

20. The device of claim 9, further comprises a power source coupled to the opposing electrodes to perform electrophoresis.
